(19) United States
(12) Patent Application Publication
(10) Pub. No.: US 2011/0190373 A1
(43) Pub. Date: Aug. 4, 2011

(54) METHODS AND COMPOSITIONS FOR THE TREATMENT OR PREVENTION OF PATHOLOGICAL CARDIAC REMODELING AND HEART FAILURE

(75) Inventors: Chen Yan, Rochester, NY (US); Jian-Dong Li, Pittsford, NY (US)

(73) Assignee: UNIVERSITY OF ROCHESTER, Rochester, NY (US)

(21) Appl. No.: 12/991,345

(22) PCT Filed: May 5, 2009
(86) PCT No.: PCT/US09/42823

§ 371 Date: Apr. 21, 2011

Related U.S. Application Data
(60) Provisional application No. 61/050,308, filed on May 5, 2008.

(51) Int. Cl. A61K 31/475; A61K 31/7088; A61K 31/522; A61P 9/00

(52) U.S. Cl. 514/44 A; 514/283; 514/263.36

(57) ABSTRACT

The invention relates to methods of treating or preventing pathological cardiac remodeling and/or preventing heart failure. These methods include the administration of a PDE1 inhibitor to a patient under conditions effective to treat or prevent pathological cardiac remodeling, and therefore heart failure that occurs as a result of such remodeling. Pharmaceutical compositions and delivery vehicles that can be used in the methods of the present invention are also disclosed herein.

This application claims the benefit of U.S. Provisional Patent Application Ser. No. 61/050,308, filed May 5, 2008, which is hereby incorporated by reference in its entirety.

### FIELD OF THE INVENTION

The present invention relates to the use of PDE1 inhibitors for treating or preventing pathological cardiac remodeling and heart failure, and pharmaceutical compositions useful for practicing these therapeutic or preventative treatments.

### BACKGROUND OF THE INVENTION

Myocyte hypertrophy, resulting from the increased size of individual cardiomyocytes, is critical for both physiological and pathological cardiac remodeling. Hypertrophy, occurring during postnatal heart development or during athletic training, is physiological hypertrophy, which does not lead to decompensated heart failure. However, excessive and sustained hypertrophy, induced by chronic mechanical and/or neurohumoral stress due to cardiovascular diseases (such as hypertension and myocardial infarction), frequently proceeds to decompensated state associated with fibrosis, myocyte death, chamber dilation, and contractile dysfunction, thereby resulting in heart failure. It is believed that pathogenic cardiac hypertrophy is a risk factor and a leading predictor of heart failure and mortality. Myocyte hypertrophic growth results from the activation of multiple signaling pathways, leading to changes in gene transcription, stimulation of protein synthesis, and increased assembly of myofibrils (Sugden et al., “Cellular Mechanisms of Cardiac Hypertrophy,” J Mol Med. 76:725-46 (1998); Molkentin et al., “Cytoplasmic Signaling Pathways that Regulate Cardiac Hypertrophy,” Annu Rev Physiol. 63:391-426 (2001). Understanding the positive and negative regulators of hypertrophic signaling pathways may lead to novel therapeutic strategies to impede pathological cardiac hypertrophy and heart failure.

It is believed that chronic neurohormonal overactivation, such as beta-adrenergic receptor (β-AR) and angiotensin II (Ang II) systems, plays a critical role in cardiac hypertrophic growth and progression to heart failure. Thus, blockade of neurohormonal activation has been considered as an important therapeutic strategy to treat and prevent pathologic cardiac remodeling. For example, β-AR antagonists, such as bisoprolol, carvedilol, and metoprolol, have been shown to significantly improve survival in heart failure patients (Waagstein et al., “Beneficial Effects of Metoprolol in Idiopathic Dilated Cardiomyopathy. Metoprolol in Dilated Cardiomyopathy (MDC) Trial Study Group,” Lancet 342:1441-6 (1993); Packer et al., “Double-blind, Placebo-controlled Study of the Effects of Carvedilol in Patients with Moderate to Severe Heart Failure. The PRECISE Trial. Prospective Randomized Evaluation of Carvedilol on Symptoms and Exercise,” Circulation 94:2793-9 (1996); Gilbert et al., “Comparative Hemodynamic, Left Ventricular Functional, and Antiadrenergic Effects of Chronic Treatment with Metoprolol Versus Carvedilol in the Failing Heart,” Circulation 94:2817-25 (1996); Packer et al., “Effect of Carvedilol on Survival in Severe Chronic Heart Failure,” N Engl J Med. 344:1651-8 (2001)). The beneficial effects of β-AR blockers on improving mortality appear to be associated with the regression of structural ventricular remodeling. Unfortunately, heart failure patients (especially with class III/IV heart failure) may not be able to tolerate β-AR blockers because of the negative inotropic effects. Therefore, there is an urgent need for developing novel therapeutic agents for prevention of pathological cardiac remodeling and progression of heart failure.

Calcium/calmodulin (Ca²⁺/CaM)-dependent signaling has been shown to stimulate myocyte gene expression and promote hypertrophic responses (Frey et al., “Decoding Calcium Signals Involved in Cardiac Growth and Function,” Nat. Med. 6:1221-7 (2000); Gruver et al., “Targeted Developmental Overexpression of Calmodulin Induces Proliferative and Hypertrophic Growth of Cardiomyocytes in Transgenic Mice,” Endocrinology 133:376-88 (1993); Colomer et al., “Chronic Elevation of Calmodulin in the Ventricles of Transgenic Mice Increases the Autonomous Activity of Calmodulin-dependent Protein Kinase II, which Regulates Atrial Natriuretic Factor Gene Expression,” Mol. Endocrinol. 14:1125-36 (2000)). Many hypertrophic stimuli, such as Ang II and adrenergic agonists, activate Ca²⁺/CaM-dependent signaling pathways. The Ca²⁺/CaM-dependent serine/threonine protein phosphatase calcineurin (CN) and Ca²⁺/CaM-dependent protein kinase II (CaMKII) are two essential effector molecules in Ca²⁺/CaM-stimulated hypertrophic responses (Wilkins et al., “Calcineurin and Cardiac Hypertrophy: Where Have We Been? Where Are We Going?” J Physiol. 541:1-8 (2002).

In contrast, cGMP signaling attenuates cardiac hypertrophy (Calderone et al., “Nitric Oxide, Atrial Natriuretic Peptide, and Cyclic GMP Inhibit the Growth-promoting Effects of Norepinephrine in Cardiac Myocytes and Fibroblasts,” J Clin Invest. 101:812-8 (1998); Silberbach et al., “Extracellular Signal-regulated Protein Kinase Activation is Required for the Anti-hypertrophic Effect of Atrial Natriuretic Factor in Neonatal Rat Ventricular Myocytes,”J Biol. Chem. 274:24858-64 (1999); Wollert et al., “Gene Transfer of cGMP-dependent Protein Kinase I Enhances the Antihypertrophic Effects of Nitric Oxide in Cardiomyocytes,” Hypertension 39:87-92 (2002); Booz, “Putting the Brakes on Cardiac Hypertrophy: Exploiting the NO-cGMP Counter-regulatory System,” Hypertension 45:341-6 (2005)). cGMP is generated by soluble and particulate guanylyl cyclases (GCs). The soluble GCs are activated by nitric oxide (NO). All three NO synthases (NOS), NOS1, 2, and 3, are expressed in the heart. Results from genetically engineered mice indicate that both NOS1 and NOS3 have anti-hypertrophic effects (Barouch et al., “Nitric Oxide Regulates the Heart by Spatial Confinement of Nitric Oxide Synthase Isoforms,” Nature 416:337-9 (2002)). Cardiac atrial (ANP) and B-type natriuretic peptide (BNP) act as local autocrine/paracrine, anti-hypertrophic and anti-fibrotic factors in the heart, through activation of the particulate guanylyl cyclase-A (GC-A) receptor and generate cGMP (Molkentin, “A Friend Within the Heart: Natriuretic Peptide Receptor Signaling,” J Clin Invest. 111:1275-7 (2003)). For example, genetic upregulation of GC-A inhibited ventricular myocyte hypertrophy in vivo (Kishimoto et al., “A Genetic Model Provides Evidence that the Receptor for Atrial Natriuretic Peptide (Guanylyl Cyclase-A) Inhibits Cardiac Ventricular Myocyte Hypertrophy,” Proc Natl Acad Sci USA 98:2703-6 (2001); Zahabi et al., “Expression of Constitutively Active Guanylate Cyclase in Cardiomyocytes Inhibits the Hypertrophic Effects of Isoproterenol and Aortic Constriction on Mouse Hearts,” J Biol. Chem. 278:47694-9 (2003)), whereas inhibition of GC-A enhanced cardiac hypertrophy (Knowles et al., “Pressure-independent Enhancement of Cardiac Hypertrophy in Natriuretic Peptide Receptor A-deficient Mice,” J Clin Invest. 107:975-84 (2001)). Expression of a cGMP downstream target, cGMP-dependent protein kinase (PKG I), attenuated cardiomyocyte hypertrophy (Wollert et al., “Gene Transfer of cGMP-dependent Protein Kinase I Enhances the Antihypertrophic Effects of Nitric Oxide in Cardiomyocytes,” Hypertension 39:87-92 (2002); Fiedler et al., “Inhibition of Calcineurin-NFAT Hypertrophy Signaling by cGMP-dependent Protein Kinase Type I in Cardiac Myocytes,” Proc Natl Acad Sci USA 99:11363-8 (2002)). These data suggest an inhibitory role for cGMP signaling in cardiac hypertrophy. Upregulation of cGMP-hydrolyzing PDE expression/activity may also contribute to the decreased cGMP signaling in diseased hearts, and inhibition of cGMP-PDE activity may enhance the anti-hypertrophic effects mediated by cGMP signaling. However, an understanding of the regulation and function of cGMP-PDE(s) in the patho-physiological remodeling of the heart is lacking.

Phosphodiesterase 1 (PDE1) family members, which are Ca²⁺/CaM-activated PDEs, play an important role in the Ca²⁺-mediated regulation of intracellular cyclic nucleotide levels due to the unique nature of Ca²⁺/CaM stimulation (Kim et al., “Upregulation of Phosphodiesterase 1A1 Expression is Associated with the Development of Nitrate Tolerance,” Circulation 104:2338-43 (2001)). The PDE1 family constitutes a large family of enzymes, and is encoded by three distinct genes, PDE1A, PDE1B and PDE1C (Rybalkin et al., “Cyclic GMP Phosphodiesterases and Regulation of Smooth Muscle Function,” Circ Res. 93:280-91 (2003)). Multiple N-terminal or C-terminal splice variants have also been identified for each gene. Currently, at least fourteen DE1A, two PDE1B, and five PDE1C transcripts have been described (Rybalkin et al., “Cyclic GMP Phosphodiesterases and Regulation of Smooth Muscle Function,” Circ Res. 93:280-91 (2003)). In vitro, the activity of all PDE1 family members can be stimulated up to 10 fold by Ca²⁺ in the presence of calmodulin (Beavo, “Cyclic Nucleotide Phosphodiesterases: Functional Implications of Multiple Isoforms,” Physiol Rev. 75:725-48 (1995)). However, they differ in their kinetic and regulatory properties, as well as tissue/cell distributions. In vitro, PDE1A and PDE1B isozymes hydrolyze cGMP with much higher affinity than cAMP, however, PDE1C isozymes hydrolyze both cAMP and cGMP with high affinity (Rybalkin et al., “Cyclic GMP Phosphodiesterases and Regulation of Smooth Muscle Function,” Circ Res. 93:280-91 (2003)). In vivo, PDE1A has been shown to preferentially hydrolyze cGMP (Hagiwara et al., “Effects of Vinpocetine on Cyclic Nucleotide Metabolism in Vascular Smooth Muscle,” Biochem Pharmacol. 33:453-7 (1984); Ahn et al., “Effects of Selective Inhibitors on Cyclic Nucleotide Phosphodiesterases of Rabbit Aorta,” Biochem Pharmacol. 38:3331-9 (1989); Nagel et al., “Role of Nuclear Ca²⁺/Calmodulin-stimulated Phosphodiesterase 1A in Vascular Smooth Muscle Cell Growth and Survival,” Circ Res. 98:777-84 (2006)). It has been found that Ca²⁺-elevating reagents such as Ang II and ET-1 rapidly activate PDE1A, leading to the attenuation of ANP- or NO-evoked cGMP accumulation in VSMCs in vitro and in vivo (Kim et al., “Upregulation of Phosphodiesterase 1A1 Expression is Associated with the Development of Nitrate Tolerance,” Circulation 104:2338-43 (2001); Jaiswal, “Endothelin Inhibits the Atrial Natriuretic Factor Stimulated cGMP Production by Activating the Protein Kinase C in Rat Aortic Smooth Muscle Cells,” Biochem Biophys Res Commun. 182:395-402 (1992); Molina et al., “Effect of in vivo Nitroglycerin Therapy on Endothelium-dependent and Independent Vascular Relaxation and Cyclic GMP Accumulation in Rat Aorta,” J Cardiovasc Pharmacol. 10:371-8 (1987)).

It has been reported that PDE1 is responsible for the majority of cGMP-hydrolyzing activity in human myocardium (Wallis et al., “Tissue Distribution of Phosphodiesterase Families and the Effects of Sildenafil on Tissue Cyclic Nucleotides, Platelet Function, and the Contractile Responses of Trabeculae Carneae and Aortic Rings in vitro,” Am J Cardiol. 83:3C-12C (1999)). However, the expression and function of PDE1 in the heart is not well documented. PDE1C expression has been detected in human heart and cardiac myocytes (Vandeput et al., “Cyclic Nucleotide Phosphodiesterase PDE1C1 in Human Cardiac Myocytes,” J Biol. Chem. 282:32749-57 (2007)), however, the function of PDE1C in human cardiomyocytes is still not clear. PDE1A mRNA expression has been described in hearts from several different species, including human (Loughney et al., “Isolation and Characterization of cDNAs Corresponding to Two Human Calcium, Calmodulin-regulated, 3′,5′-cyclic Nucleotide Phosphodiesterases,” J Biol. Chem. 271:796-806 (1996)), cow (Sonnenburg et al., “Molecular Cloning of a cDNA Encoding the ‘61-kDa’ Calmodulin-stimulated Cyclic Nucleotide Phosphodiesterase. Tissue-specific Expression of Structurally Related Isoforms,” J Biol. Chem. 268:645-52 (1993)), dog (Clapham et al., “Cloning of Dog Heart PDE1A—A First Detailed Characterization at the Molecular Level in this Species,” Gene 268:165-71 (2001)), and rat (Yanaka et al., “cGMP-phosphodiesterase Activity is Up-regulated in Response to Pressure Overload of Rat Ventricles,” Biosci Biotechnol Biochem. 67:973-9 (2003)). Because most of these studies utilized whole hearts, it is unclear if these isoforms are attributed to cardiomyocytes or other cell types existing in the heart.

From the foregoing, it remains unclear what role PDE1 may play in pathological cardiac remodeling and heart failure, and whether inhibitors of PDE1 isoforms can be used alone or in combination with other therapeutic agents to treat or prevent pathological cardiac remodeling and inhibit the progression of heart failure.

The present invention is directed to overcoming these and other deficiencies in the art.

### SUMMARY OF THE INVENTION

A first aspect of the present invention relates to a method of treating or preventing pathological cardiac remodeling that includes: providing an inhibitor of PDE1 activity (“PDE1 inhibitor”); and administering the PDE1 inhibitor to a patient under conditions effective to treat or prevent pathological cardiac remodeling.

A second aspect of the present invention relates to a method of preventing heart failure that includes: providing a PDE1 inhibitor; and administering the PDE1 inhibitor to a patient susceptible to pathological cardiac remodeling under conditions effective to prevent heart failure caused by pathological cardiac remodeling.

A third aspect of the present invention relates to a pharmaceutical composition that includes a PDE1 inhibitor and either a β-blocker, a β-agonist, a PDE3 inhibitor, a metabolism-boosting agent, or a combination thereof. The pharmaceutical composition may also include an angiotensin II receptor (type 1) antagonist and/or an angiotensin-converting enzyme (“ACE”) inhibitor.

A fourth aspect of the present invention relates to a therapeutic system for treatment of pathologic cardiac remodeling that includes a PDE1 inhibitor and either a β-blocker, a β-agonist, a PDE3 inhibitor, a metabolism-boosting agent, or a combination thereof, and may further include an angiotensin II receptor (type 1) antagonist and/or an ACE inhibitor.

A fifth aspect of the present invention relates to a delivery vehicle that includes a pharmaceutical composition of the invention. The delivery vehicle can be in any form, but preferably in the form of a transdermal patch, a syringe, or a biocompatible polymeric matrix.

The inventors have recently discovered that both PDE1A and PDE1C mRNA and protein were detected in human hearts, and PDE1A expression was conserved in rodent hearts (such as rat and mouse hearts). PDE1A expression was significantly upregulated in vivo in the heart from various pathological hypertrophy animal models and in vitro in isolated rat neonatal and adult cardiomyocytes treated with neurohumoral stimuli such as Ang II and isoproterenol (ISO) Inhibition of PDE1 activity using PDE1 inhibitors (such as 8mM-IBMX and vinpocetine) significantly abrogated ISO or phenylephrine (PE) induced pathological myocyte hypertrophy and hypertrophic marker expression. Downregulation of PDE1A using siRNA also significantly abrogated PE induced cardiomyocyte hypertrophy and hypertrophic marker expression. These results demonstrate that PDE1, particularly PDE1A, plays a crucial role in regulating cardiomyocyte hypertrophic growth, and pathological upregulation of PDE1A may contribute to the progression of cardiac hypertrophy and remodeling. Vinpocetine, a known PDE1 inhibitor, significantly attenuated cardiac hypertrophy in isolated cardiomyocytes and in a mouse model of cardiac hypertrophy induced by chronic ISO infusion.

These examples presented herein identify PDE1 as a novel therapeutic target for cardiac hypertrophy Inhibition of PDE1 with vinpocetine or other PDE1 inhibitors will reduce pathological myocyte hypertrophy and prevent subsequent heart failure. Given that vinpocetine has already been clinically approved to be safe, vinpocetine is an ideal therapeutic agent for prevention of pathological cardiac remodeling and progression of heart failure. Based on the foregoing, the present invention identifies a new therapeutic strategy for the treatment of cardiac remodeling and failure.

### BRIEF DESCRIPTION OF THE DRAWINGS

FIG. 1A-F show PDE1 family enzyme expression in the heart and isolated cardiomyocytes. FIGS. 1A-C illustrate RT-PCR results showing PDE1A, PDE1B, and PDE1C mRNA expression in adult human, rat, and mouse heart tissue compared to indicated controls (mouse brain for PDE1A and 1B or mouse testis for PDE1C). RT-PCR data was quantified by densitometry in a linear range from three independent samples, which were normalized to GAPDH mRNA levels and expressed relative to human hearts (AU=arbitrary units). FIG. 1D is a representative Western blot showing relative PDE1A, PDE1B, and PDE1C protein levels in human, rat, and mouse hearts, compared to respective controls (brain for PDE1A and PDE1B; testis for PDE1C). GAPDH was used to normalize protein loading. FIG. 1E illustrates RT-PCR results showing relative PDE1A, 1B, and 1C mRNA levels in neonatal rat ventricular myocyte (NRVM), rat adult ventricular myocyte (ARVM), and rat hearts, compared to respective controls. FIG. 1F is a Western blot depicting relative PDE1A, 1B, and 1C protein levels in NRVM and ARVM compared to rat hearts and respective controls. GAPDH was used to normalize mRNA and protein expression.

FIG. 2A-E show that PDE1A expression is upregulated with cardiac hypertrophy both in vivo and in vitro. FIG. 2A is a Western blot showing PDE1A protein levels in ventricular tissues from mice subjected to chronic vehicle or ISO infusion (30 mg/kg/d) for 7 days. FIG. 2B is a Western blot showing PDE1A protein levels in ventricular tissues from mice subjected to pressure overload by TAC or sham operation for 4 weeks. FIG. 2C is a Western blot showing PDE1A protein levels in ventricular tissues from rats subjected to vehicle or chronic Ang II infusion (0.7 mg/kg/d) for 14 days. FIGS. 2D-E are Western blots showing PDE1A protein expression in isolated NRVM treated with ISO (10 mmol/L) or vehicle (ctrl) for up to 48 hours (FIG. 2D), or in ARVM treated with ISO (1 mmol/L), Ang II (100 μmol/L), or vehicle (ctrl) for 24 hours (FIG. 2E).

FIGS. 3A-C show the effects of PDE1 inhibitors on pathological cardiomyocyte hypertrophy. Myocyte hypertrophy was induced in NRVM by α-adrenergic agonist, phenylephrine (PE). Hypertrophy was assessed by protein synthesis by measuring [³H]-leucine incorporation (normalized to the total DNA content), or by myocyte surface area. PDE inhibitor 8-MM-IBMX (at 10 μM the concentration selective to PDE1) blocked PE-induced cardiomyocyte protein synthesis measured by [³H]-leucine incorporation (FIG. 3A) or measured by myocyte surface area (FIG. 3B). Vinpocetine (20 μM), known as PDE1 inhibitor, also significantly blocked PE-induced hypertrophy measured by myocyte surface area (FIG. 3C). These results demonstrate that PDE1 activity plays a critical role in the cardiomyocyte hypertrophic growth.

FIG. 4A-D show the effects of PDE1A knock-down by PDE1A siRNA (encoding DNA TGTCAACGTTGTCGACCTA, SEQ ID NO: 1) on cardiomyocyte hypertrophy. As shown in FIG. 4A, PDE1A siRNA significantly down-regulated PDE1A protein expression compared with the control siRNA. As expected, PDE1A siRNA significantly blocked PE-induced cardiomyocyte hypertrophy measured by the cell surface area or [³H]-leucine incorporation (FIG. 4B) and myocyte surface area (FIG. 4C). Consistently, PDE1A siRNA also blocked PE-induced hypertrophic gene ANP mRNA expression measured by RT-PCR (FIG. 4D). These results demonstrate that PDE1A is likely involved in mediating a hypertrophic response in cardiomyocytes.

FIGS. 5A-F illustrate that Vinpocetine attenuates cardiac hypertrophy in vivo. C57 mice received continuous vehicle (0.002% ascorbic acid in PBS) or ISO (30 mg/kg/d) infusion via osmotic pumps for 7 days, and also received daily DMSO or Vinpocetine treatment (i.p. 10 mg/kg/d). Control group (Con): mice receiving only vehicle infusion for 7 days. ISO group: mice receiving ISO infusion and DMSO treatment for 7 days. ISO+Vinp group: mice receiving ISO infusion and vinpocetine treatment for 7 days. After 7 days, animals were sacrificed and hearts were excised, weighed, frozen in −80° C. for mRNA assay, or fixed in 10% formalin for histology analysis. FIG. 5A are representative gross heart images showing effects of PDE1 inhibitor on cardiac hypertrophy. FIGS. 5B-C are graphs showing the effect of Vinpocetine on heart to body weight ratio or heart weight to tibial length ratio, respectively. FIG. 5D shows a comparison of left ventricle cross-sections from the control mice (left panel), ISO-infused and DMSO treated mice (middle panel), and ISO-infused and Vinpocetine treated mice (right panel) (magnification X200). FIGS. 5E-F are graphs showing the effect of Vinpocetine on ANP and BNP mRNA expression, respectively. Total RNA from left ventricles were subjected to real-time RT-PCR analyses for the mRNA levels of ANP and BNP. Data were normalized to control and sham samples that were arbitrarily set to 1.0. Data represent mean of 4 animals (mean±SEM). **P<0.01 vs. control mice. ##P<0.01 vs. ISO-infused mice without Vinpocetine.

### DETAILED DESCRIPTION OF THE INVENTION

The present invention relates to methods of treating or preventing pathological cardiac remodeling and preventing heart failure. These methods include the administration of a PDE1 inhibitor to a patient under conditions effective to treat or prevent pathological cardiac remodeling, and therefore heart failure that occurs as a result of such remodeling. Pharmaceutical compositions and delivery vehicles that can be used in the methods of the present invention are also disclosed herein.

As used herein, the patient to be treated can be any mammal, but preferably the mammal is a human, a non-human primate, a rodent, a cow, a horse, a sheep, or a pig. Other mammals can also be treated in accordance with the present invention.

As used herein, the term “pathological cardiac remodeling” is intended to encompass any alteration of cellular structure of cardiac myocytes or fibroblasts, or alteration of cardiac tissue structure, morphology, and function resembling cardiomyopathy. These alterations of cardiac cellular or tissue structure can include, without limitation, cell death (either apoptotic or necrotic cell death), fibrosis, and/or myocyte hypertrophy and elongation.

The PDE1 inhibitor can be any suitable inhibitor of PDE1 isoforms, including PDE1A inhibitor, PDE1B inhibitors, PDE1C inhibitors, or inhibitors of multiple PDE1 inhibitors (pan-PDE1 inhibitors). Exemplary PDE1 inhibitors include, without limitation, bepridil, flunarizine, amiodarone, 8-MM-IBMX, IC86340, IC295, compounds from Kyowa Hakko Kogyo Co. Ltd. including KS-505a, K-295-2, and KS-619-1, compounds from Schering-Plough Research Institute including SCH51866, SCH45752 (Cephalochromin), and compounds 30 and 31 (Dunkern et al., “Characterization of Inhibitors of Phosphodiesterase 1C on a Human Cellular System,” FEBS J. 274(18):4812-24 (2007), which is hereby incorporated by reference in its entirety), a vincamine derivative, a ginsenoside, and anti-PDE1 antisense oligos and RNAi, including both microRNA (miRNA), small interfering RNA (siRNA), and small hairpin RNA (shRNA).

Activity of these or other agents as PDE1 inhibitors can be assessed using known in vitro PDE1 activity assays. Basically, PDE1 (0.75 mU) and CaCl₂ (0.2 mM) are incubated at 30° C. for 10 min in 0.3 ml of a reaction buffer containing 50 mM HEPES-NaOH (pH 7.5), 0.1 mM EGTA, 8.3 mM MgCl₂, 0.5 μM [³H]cAMP (18,000 cpm) and any agent being tested for PDE1 inhibition. This is performed in parallel, with and without CaM (10 mU). PDE1 activity in the presence and absence of the agent being tested can be assayed using the procedures described in Shimizu et al., “Calmodulin-Dependent Cyclic Nucleotide Phosphodiesterase (PDE1) Is a Pharmacological Target of Differentiation-Inducing Factor-1, an Antitumor Agent Isolated from Dictyostelium,” Cancer Research 64:2568-2571 (2004); Murata et al., “Differential Expression of cGMP-Inhibited Cyclic Nucleotide Phosphodiesterases in Human Hepatoma Cell Lines,” FEBS Lett, 390:29-33 (1996), each of which is hereby incorporated by reference in its entirety.

In an alternative assay, PDE activity can be determined using 1 μM cyclic nucleotide as substrate via a two-step radioassay procedure adapted from Thompson and Appleman, “Characterization of Cyclic Nucleotide Phosphodiesterases of Rat Tissues,” J Biol Chem 246:3145-3150 (1971); Murray et al., “Expression and Activity of cAMP Phosphodiesterase Isoforms in Pulmonary Artery Smooth Muscle Cells from Patients with Pulmonary Hypertension: Role for PDE1,” Am J Physiol Lung Cell Mol Physiol 292:L294-L303 (2007), each of which is hereby incorporated by reference in its entirety. Briefly, substrate and protein sample can be incubated over a period of time that PDE1 activity is linear (e.g., 30 min), after which they can be boiled for 2 min to terminate the reaction. Assays can be performed in the presence or absence of putative PDE inhibitors being screened, and with or without calcium in the presence of EGTA.

Suitable vincamine derivative can be any known or hereafter developed derivative of vincamine that has an inhibitory activity on any PDE1 isoforms, but preferably on the PDE1A isoforms.

Vincamine has the structure

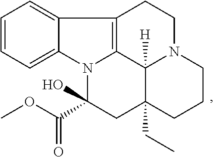

and its recovery from the leaves of Vinca minor L. is well known in the art. A number of vincamine derivatives have been synthesized and are well tolerated for therapeutic administration.

Exemplary vincamine derivatives include, without limitation:

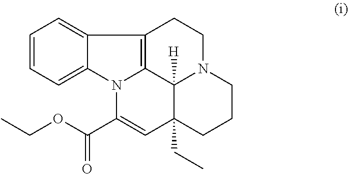

(+)-vinpocetine or salts thereof;

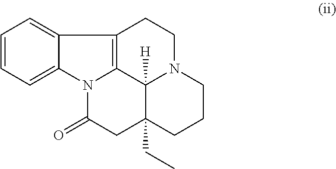

(−)-eburnamonine (also known as viburnine) or salts thereof;

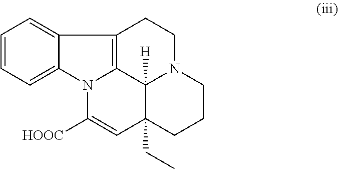

apovincaminic acid or salts thereof;

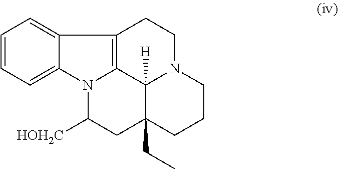

(3S,16R)-didydro-eburnamenine-4-methanol (also known as RGH-0537) or salts thereof;

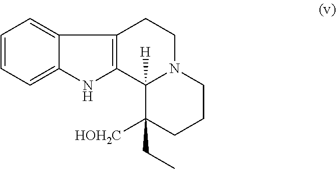

(1S,12S)-indoloquinolizinyl-1-methanol (also known as RGH-2981 or vintoperol) or salts thereof;

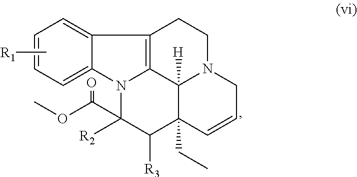

where R₁ is a halogen, R₂ can be a hydroxy group whereas R₃ can be hydrogen, or R₂ and R₃ together form an additional bond between the carbon atoms which carry them, or salts thereof;

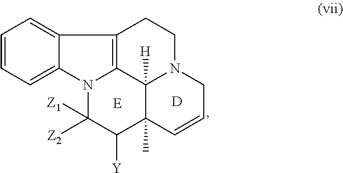

where the compound is formed by a cis-fusion of the D/E rings, and either (i) Y is hydrogen, in which case Z₁ and Z₂ together represent simultaneously an oxygen atom or Z₁ is a methoxycarbonyl radical and Z₂ is a hydroxy radical, or (ii) where Y and Z₂ together form a carbon-carbon bond and Z₁ is a methoxycarbonyl radical, or salts thereof;

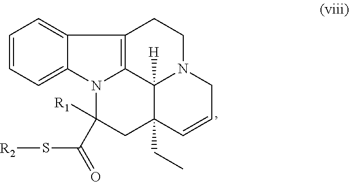

where R₁ is hydrogen or a hydroxyl group, and R₂ is an alkyl group, or salts thereof;

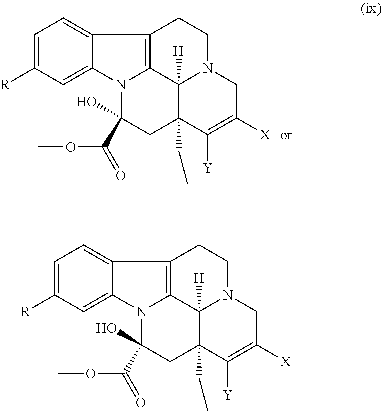

where R is hydrogen or methoxy, X and Y are hydrogen or are together are a double bond between the ring carbon atoms to which they are bonded, or salts thereof; and (x) combinations of any two or more of the above compounds or salts thereof.

Vinpocetine is produced by slightly altering the vincamine molecule, an alkaloid extracted from the Periwinkle plant, Vinca minor. Vinpocetine was originally discovered and marketed in 1978 under the trade name Vavinton (Hungary). Since then, Vinpocetine has been widely used in many countries for preventative treatment of cerebrovascular disorder and cognitive impairment including stroke, senile dementia, and memory disturbances due to the beneficial cerebrovascular effect and neuroprotective profile (Bonoczk et al., “Role of Sodium Channel Inhibition in Neuroprotection: Effect of Vinpocetine,” Brain Res Bull. 53:245-54 (2000), which is hereby incorporated by reference in its entirety). For instance, different types of vinpocetine-containing memory enhancer (named Intelectol® in Europe, and Memolead® in Japan) have been currently used as a dietary supplement worldwide. Vinpocetine is a cerebral vasodilator that improves brain blood flow (Bonoczk et al., “Role of Sodium Channel Inhibition in Neuroprotection: Effect of Vinpocetine,” Brain Res Bull. 53:245-54 (2000), which is hereby incorporated by reference in its entirety). Vinpocetine has also been shown to act as a cerebral metabolic enhancer by enhancing oxygen and glucose uptake from blood and increasing neuronal ATP bio-energy production (Bonoczk et al., “Role of Sodium Channel Inhibition in Neuroprotection: Effect of Vinpocetine,” Brain Res Bull. 53:245-54 (2000), which is hereby incorporated by reference in its entirety). Vinpocetine appears to have multiple cellular targets such as Ca²⁺/Calmodulin-stimulated phosphodiesterases (PDE1), and voltage-dependent Na⁺-channels and Ca²⁺-channels (Bonoczk et al., “Role of Sodium Channel Inhibition in Neuroprotection: Effect of Vinpocetine,” Brain Res Bull. 53:245-54 (2000), which is hereby incorporated by reference in its entirety). To date, there have been no reports of significant side effects, toxicity or contraindications at the therapeutic doses (Balestreri et al., “A double-blind Placebo Controlled Evaluation of the Safety and Efficacy of Vinpocetine in the Treatment of Patients with Chronic Vascular Senile Cerebral Dysfunction,” J Am Geriatr Soc. 35:425-30 (1987), which is hereby incorporated by reference in its entirety). Because of these reasons, vinpocetine has long been thought as an interesting compound that constantly attracts scientists and clinicians to seek its novel therapeutic application as well as its underlying molecular mechanisms.

The compounds can also be in the form of a salt, preferably a pharmaceutically acceptable salt. The term “pharmaceutically acceptable salt” refers to those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present invention.

It should also be appreciated that other vincamine derivatives can also be used in accordance with the present invention. These include the peripherally active vincamine derivatives, such as RGH-0537 and RGH-2981, both identified above. In other embodiment, those vincamine derivatives capable of crossing the blood-brain barrier can be used, such as vinpocetine.

The inhibitor of PDE1 can also take the form of a gene-silencing oligonucleotide known as RNA-interference (RNAi), which utilizes an antisense molecule that interferes with endogenous PDE1 isoform expression. RNAi is a form of post-transcriptional gene silencing (PTGS) via introduction of a homologous double-stranded RNA (dsRNA), transgene, or virus. In PTGS, the transcript of the silenced gene is synthesized, but does not accumulate because it is degraded. RNAi is a specific from of PTGS, in which the gene silencing is induced by the direct introduction of dsRNA. Numerous reports have been published on critical advances in the understanding of the biochemistry and genetics of both gene silencing and RNAi (Matzke et al., “RNA-Based Silencing Strategies in Plants,” Curr Opin Genet Dev 11(2):221-227 (2001); Hammond et al., “Post-Transcriptional Gene Silencing by Double-Stranded RNA,” Nature Rev Gen 2:110-119 (2001); Hamilton et al., “A Species of Small Antisense RNA in Posttranscriptional Gene Silencing in Plants,” Science 286:950-952 (1999); Hammond et al., “An RNA-Directed Nuclease Mediates Post-Transcriptional Gene Silencing in Drosophila Cells,” Nature 404:293-298 (2000); Hutvagner et al., “RNAi: Nature Abhors a Double-Strand,” Curr Opin Genetics & Development 12:225-232 (2002), each of which is hereby incorporated by reference in its entirety). In iRNA, the introduction of double stranded RNA (dsRNA) into cells leads to the destruction of the endogenous, homologous mRNA, phenocopying a null mutant for that specific gene. In siRNA, the dsRNA is processed to short interfering molecules of 21-, 22- or 23-nucleotide RNAs (siRNA), which are also called “guide RAs,” (Hammond et al., “Post-Transcriptional Gene Silencing by Double-Stranded RNA,” Nature Rev Gen 2:110-119 (2001); Sharp, P. A., “RNA Interference-2001,” Genes Dev 15:485-490 (2001); Hutvagner et al., “RNAi: Nature Abhors a Double-Strand,” Curr Opin Genetics & Development 12:225-232 (2002), each of which is hereby incorporated by reference in its entirety) in vivo by the Dicer enzyme, a member of the RNAse III-family of dsRNA-specific ribonucleases (Hutvagner et al., “RNAi: Nature Abhors a Double-Strand,” Curr Opin Genetics & Development 12:225-232 (2002); Bernstein et al., “Role for a Bidentate Ribonuclease in the Initiation Step of RNA Interference,” Nature 409:363-366 (2001); Tuschl, “RNA Interference and Small Interfering RNAs,” Chembiochem 2:239-245 (2001); Zamore et al., “RNAi: Double Stranded RNA Directs the ATP-Dependent Cleavage of mRNA at 21 to 23 Nucleotide Intervals,” Cell 101:25-3 (2000); U.S. Pat. No. 6,737,512 to Wu et al., each of which is hereby incorporated by reference in its entirety). Successive cleavage events degrade the RNA to 19-21 bp duplexes, each with 2-nucleotide 3′ overhangs (Hutvagner et al., “RNAi: Nature Abhors a Double-Strand,” Curr Opin Genetics & Development 12:225-232 (2002); Bernstein et al., “Role for a Bidentate Ribonuclease in the Initiation Step of RNA Interference,” Nature 409:363-366 (2001), each of which is hereby incorporated by reference in its entirety). The siRNAs are incorporated into an effector known as the RNA-induced silencing complex (RISC), which targets the homologous endogenous transcript by base pairing interactions and cleaves the mRNA approximately 12 nucleotides form the 3′ terminus of the siRNA (Hammond et al., “Post-Transcriptional Gene Silencing by Double-Stranded RNA,” Nature Rev Gen 2:110-119 (2001); Sharp, P. A., “RNA Interference-2001,” Genes Dev 15:485-490 (2001); Hutvagner et al., “RNAi: Nature Abhors a Double-Strand,” Curr Opin Genetics & Development 12:225-232 (2002); Nykanen et al., “ATP Requirements and Small Interfering RNA Structure in the RNA Interference Pathway,” Cell 107:309-321 (2001), each of which is hereby incorporated by reference in its entirety).

There are several methods for preparing siRNA, including chemical synthesis, in vitro transcription, siRNA expression vectors, and PCR expression cassettes. In one aspect of the present invention, dsRNA for the nucleic acid molecule of the present invention can be generated by transcription in vivo. This involves modifying the nucleic acid molecule of the present invention for the production of dsRNA, inserting the modified nucleic acid molecule into a suitable expression vector having the appropriate 5′ and 3′ regulatory nucleotide sequences operably linked for transcription and translation, as described above, and introducing the expression vector having the modified nucleic acid molecule into a suitable host or subject. Using siRNA for gene silencing is a rapidly evolving tool in molecular biology, and guidelines are available in the literature for designing highly effective siRNA targets and making antisense nucleic acid constructs for inhibiting endogenous protein (U.S. Pat. No. 6,737,512 to Wu et al.; Brown et al., “RNA Interference in Mammalian Cell Culture: Design, Execution, and Analysis of the siRNA Effect,” Ambion TechNotes 9(1):3-5 (2002); Sui et al., “A DNA Vector-Based “RNAi Technology to Suppress Gene Expression in Mammalian Cells,” Proc Natl Acad Sci USA 99(8):5515-5520 (2002); Yu et al., “RNA Interference by Expression of Short-Interfering RNAs and Hairpin RNAs in Mammalian Cells,” Proc Natl Acad Sci USA 99(9): 6047-6052 (2002); Paul et al., “Effective Expression of Small Interfering RNA in Human Cells,” Nature Biotechnology 20:505-508 (2002); Brummelkamp et al., “A System for Stable Expression of Short Interfering RNAs in Mammalian Cells,” Science 296:550-553 (2002), each of which is hereby incorporated by reference in its entirety). There are also commercially available sources for custom-made siRNAs.

Exemplary siRNA and shRNA inhibitors of PDE1A include, without limitation, those encoded by:

(SEQ ID NO: 1 for siRNA)
TGTCAACGTTGTCGACCTA;
and
(SEQ ID NO: 2 for shRNA)
GAACTTGATCTTCATAAGAACTCAGAAGA.

A number of other PDE1A, PDE1B, and PDE1C RNAi are available from Santa Cruz Biotechnology, Ltd., Ambion Inc., and other suppliers. Any other siRNA and shRNA inhibitors, or full length or near-full length antisense RNA molecules of PDE1A, PDE1B, or PDE1C can also be employed herein.

RNAi-encoding genes can be prepared using well-known recombinant molecular techniques, which includes ligating the RNAi-specific sequence to its appropriate regulatory regions using well known molecular cloning techniques (Sambrook et al., Molecular Cloning: A Laboratory Manual, Second Edition, Cold Spring Harbor Press, NY (1989), which is hereby incorporated by reference in its entirety). The recombinant gene can then be introduced into a suitable vector or otherwise introduced directly into a host cell using transformation protocols well known in the art. For example, cardiomyocyte-specific expression of the recombinant gene can be achieved by using the cardiac muscle-specific alpha myosin heavy chain (MHC) gene promoter and a recombinant adeno-associated viral vector to deliver the gene (Aikawa et al., “Cardiomyocyte-specific Gene Expression Following Recombinant Adeno-associated Viral Vector Transduction,” J. Biol. Chem. 277(21):18979-18985 (2002), which is hereby incorporated by reference in its entirety).

Both therapeutic and preventative use of the PDE1 inhibitors is contemplated herein.

According to one embodiment, administration of the PDE1 inhibitor is intended to be used to treat symptoms of pre-existing pathological cardiac remodeling. In this case, the patient to be treated can be symptomatic for heart failure, i.e., in any of phases I to IV of heart failure. Administration of the PDE1 inhibitor can be effective to inhibit the progression of heart failure symptoms, reduce the rate of progression of heart failure symptoms, or reverse the severity of heart failure symptoms. Under these conditions, it may also be desirable to administer to the patient a β-agonist or an inhibitor of phosphodiesterase 3 activity (a PDE3 inhibitor).

According to another embodiment, administration of the PDE1 inhibitor is intended to be used to prevent onset of cardiac remodeling. For example, post-myocardial infarction patients can be administered PDE1 inhibitors to prevent subsequent remodeling. This can protect against heart failure or resist progression of the disease. Under these conditions, it may also be desirable to administer to the patient a β-blocker.

Thus, the present invention contemplates co-administering with the PDE1 inhibitor a therapeutically effective amount of an additional therapeutic agent. The additional therapeutic agent can be selected from the group of β-blockers, β-agonists, a PDE3 inhibitor, an angiotensin II receptor (type 1) antagonist, an angiotensin-converting enzyme (ACE) inhibitor, a metabolism-boosting agent, and combinations of any two or more of these additional therapeutic agents.

β-AR antagonists (β-blockers) are known to improve survival in heart failure patients significantly. Although the favorable effects of β-AR blockers on mortality appear to be associated with the regression of structural ventricular remodeling, phase III/IV heart failure patients may not be able to tolerate β-AR blockers because of the negative inotropic effects. Any suitable β-blocker can be administered in combination with the PDE1 inhibitor.

Exemplary β-blockers include, without limitation, acebutolol, atenolol, betaxolol, bisoprolol or bisoprolol fumarate, carvedilol, carteolol, celeprolol, esmolol or esmolol hydrochloride, labetalol, metoprolol or metoprolol succinate or metoprolol tartrate, nadolol, nebivolol, oxprenolol, penbutolol, pindolol, propranolol or propranolol hydrochloride, sotalol, esmolol, timolol, bopindolol, medroxalol, bucindolol, levobunolol, metipranolol, celiprolol, propafenone, and combinations thereof.

β-AR agonists (β-agonists) are known to afford great acute beneficial effects in patients with early stage heart failure due to their inotropic effects, although their use is typically short term due to increased mortality in patients receiving chronic treatment. Any suitable β-agonist can be administered in combination with the PDE1 inhibitor.

Exemplary β-agonists include, without limitation, dobutamine, formoterol or formoterol fumarate, fenoterol, ritodrin, salbutinol, terbutaline, isoproterenol, clenbuterol, and combinations thereof.

PDE3 inhibitors have shown similar efficacy and side effects to β-agonists; thus, there use of similarly limited to short term use during early stages of heart failure. Any suitable PDE3 inhibitor can be administered in combination with the PDE1 inhibitor.

Exemplary PDE3 inhibitors include, without limitation, milrinone, aminone, enoximone, and combinations thereof.

Because acute beneficial and chronic detrimental effects of cAMP (via β-ARagonists) are mediated by different molecular mechanisms, vinpocetine may block the detrimental effects of β-AR agonists and PDE3 inhibitors. Thus, the combination of β-agonist or PDE3 inhibitor with Vinpocetine is expected to be quite effective.

The mechanism of action for ACE inhibitors is via an inhibition of angiotensin-converting enzyme (ACE) that prevents conversion of angiotensin Ito angiotensin II, a potent vasoconstrictor, resulting in lower levels of angiotensin II, which causes a consequent increase in plasma renin activity and a reduction in aldosterone secretion. Angiotensin Receptor Blockers (ARBs) work as their name implies by directly blocking angiotensin II receptors and thus preventing the action of angiotensin II.

The term ACE inhibitor is intended to embrace any agent or compound, or a combination of two or more agents or compounds, having the ability to block, partially or completely, the rapid enzymatic conversion of the physiologically inactive decapeptide form of angiotensin (“Angiotensin I”) to the vasoconstrictive octapeptide form of angiotensin (“Angiotensin II”).

Examples of suitable ACE inhibitors include, without limitation, the following compounds: AB-103, ancovenin, benazeprilat, BRL-36378, BW-A575C, CGS-13928C, CL242817, CV-5975, Equaten, EU4865, EU-4867, EU-5476, foroxymithine, FPL 66564, FR-900456, Hoe-065, 15B2, indolapril, ketomethylureas, KR1-1177, KR1-1230, L681176, libenzapril, MCD, MDL-27088, MDL-27467A, moveltipril, MS41, nicotianamine, pentopril, phenacein, pivopril, rentiapril, RG-5975, RG-6134, RG-6207, RGH0399, ROO-911, RS-10085-197, RS-2039, RS 5139, RS 86127, RU-44403, S-8308, SA-291, spiraprilat, SQ26900, SQ-28084, SQ-28370, SQ-28940, SQ-31440, Synecor, utibapril, WF-10129, Wy-44221, Wy-44655, Y-23785, Yissum, P-0154, zabicipril, Asahi Brewery AB-47, alatriopril, BMS182657, Asahi Chemical C-111, Asahi Chemical C-112, Dainippon DU-1777, mixanpril, Prentyl, zofenoprilat, I (-(1-carboxy-6-(4-piperidinyl)hexyl)amino)-1-oxo-propyl octahydro-1H-indole-2-carboxylic acid, Bioproject BP1.137, Chiesi CHF 1514, Fisons FPL-66564, idrapril, perindoprilat and Servier S-5590, alacepril, benazepril, captopril, cilazapril, delapril, enalapril, enalaprilat, fosinopril, fosinoprilat, imidapril, lisinopril, perindopril, quinapril, ramipril, ramiprilat, saralasin acetate, temocapril, tranolapril, trandolaprilat, ceranapril, moexipril, quinaprilat spirapril, and combinations thereof.

The term “ACE inhibitor” also embraces so-called NEP/ACE inhibitors (also referred to as selective or dual acting neutral endopeptidase inhibitors) which possess neutral endopeptidase (NEP) inhibitory activity and angiotensin converting enzyme (ACE) inhibitory activity. Examples of NEP/ACE inhibitors include those disclosed in U.S. Pat. Nos. 5,508,272 to Robl, 5,362,727 to Robl, 5,366,973 to Flynn et al., 5,225,401 to Seymour, 4,722,810 to Delaney et al., 5,223,516 to Delaney et al., 5,552,397 to Karanewsky et al., 4,749,688 to Haslanger et al., 5,504,080 to Karanewsky, 5,612,359 to Murugesan, 5,525,723 to Robl, 5,430,145 to Flynn et al., and 5,679,671 to Oinuma et al., as well as European Patent Applications 0481522 to Flynn et al., 0534263 to Pietro et al., 0534396 to Warshawsky et al., 0534492 to Warshawsky et al., and 0671172 to Oinuma et al., each of which is hereby incorporated by reference in its entirety. Especially preferred is the NEP/ACE inhibitor omapatrilat (disclosed in U.S. Pat. No. 5,508,272) or MDL100240 (disclosed in U.S. Pat. No. 5,430,145).

The term “angiotensin II receptor (type 1) antagonist” is intended to embrace any agent or compound, or a combination of two or more agents or compounds, having the ability to block, partially or completely the binding of angiotensin II at angiotensin receptors, specifically at the AT₁ receptor. These agents are also known as Angiotension Receptor Blockers (ARBs).

Examples of suitable angiotensin II antagonists include, without limitation, the following compounds: saralasin acetate, candesartan cilexetil, CGP-63170, EMD-66397, KT3-671, LR-B/081, valsartan, A-81282, BIBR-363, BIBS-222, BMS-184698, candesartan, CV-11194, EXP-3174, KW-3433, L-161177, L-162154, LR-B/057, LY-235656, PD-150304, U-96849, U-97018, UP-275-22, WAY-126227, WK-1492.2K, YM-31472, losartan potassium, E-4177, EMD-73495, eprosartan, HN-65021, irbesartan, L-159282, ME-3221, SL-91.0102, Tasosartan, Telmisartan, UP-269-6, YM-358, CGP-49870, GA-0056, L-159689, L-162234, L-162441, L-163007, PD-123177, A-81988, BMS-180560, CGP-38560A, CGP48369, DA-2079, DE-3489, DuP-167, EXP-063, EXP-6155, EXP-6803, EXP-7711, EXP-9270, FK-739, HR-720, ICI-D6888, ICI-D7155, ICI-D8731, isoteoline, KR1-1177, L-158809, L-158978, L-159874, LR B087, LY-285434, LY-302289, LY-315995, RG-13647, RWJ-38970, RWJ-46458, S-8307, S-8308, saprisartan, saralasin, sarmesin, WK-1360, X-6803, ZD-6888, ZD-7155, ZD-8731, BIBS39, C1-996, DMP-811, DuP-532, EXP-929, L-163017, LY-301875, XH-148, XR-510, zolasartan, PD-123319, and combinations thereof.

Any suitable metabolism-boosting agent can also be co-administered with the PDE1 inhibitor. The metabolism-boosting agent is intended to promote cardiomyocyte function, which should improve cardiac function. Exemplary metabolism-boosting agents include, without limitation, coenzyme A, ATP, coenzyme Q10 (CQ10), NAD(P)H, insulin-like growth factor-1 (IGF-1), and combinations thereof.

Preferred pharmaceutical compositions of the present invention include, without limitation, an effective amount of a PDE1 inhibitor in combination with an effective amount of a β-blocker; an effective amount of a PDE1 inhibitor in combination with an effective amount of a β-agonist or a PDE3 inhibitor; an effective amount of a PDE1 inhibitor in combination with an effective amount of a metabolism-boosting agent; or combinations of an effective amount of a PDE1 inhibitor with effective amounts of two or more of a β-blocker, a β-agonist or a PDE3 inhibitor, a metabolism-boosting agent, an ACE inhibitor, and an angiotensin II antagonist.

Exemplary modes of administration include, without limitation, orally, by inhalation, by airway instillation, optically, intranasally, topically, transdermally, parenterally, subcutaneously, intravenous injection, intra-arterial injection, intradermal injection, intramuscular injection, intrapleural instillation, intraperitoneal injection, intracardiac injection, intraventricularly, intralesionally, by application to mucous membranes, or implantation of a sustained release vehicle.

The PDE1 inhibitor can be administered alone or the additional therapeutic agents can be co-administered either in a single formulation or separately as multiple doses. Administration is preferably carried out via the above routes.

These active agents are preferably administered in the form of pharmaceutical formulations that include one or more of the active agents together with a pharmaceutically acceptable carrier. The term “pharmaceutically acceptable carrier” refers to any suitable adjuvants, carriers, excipients, or stabilizers, and can be in solid or liquid form such as, tablets, capsules, powders, solutions, suspensions, or emulsions.

Typically, the composition will contain from about 0.01 to 99 percent, preferably from about 20 to 75 percent of active compound(s), together with the adjuvants, carriers and/or excipients.

The solid unit dosage forms can be of the conventional type. The solid form can be a capsule and the like, such as an ordinary gelatin type containing the compounds of the present invention and a carrier, for example, lubricants and inert fillers such as, lactose, sucrose, or cornstarch. In another embodiment, these compounds are tableted with conventional tablet bases such as lactose, sucrose, or cornstarch in combination with binders like acacia, cornstarch, or gelatin, disintegrating agents, such as cornstarch, potato starch, or alginic acid, and a lubricant, like stearic acid or magnesium stearate.

The tablets, capsules, and the like can also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it can contain, in addition to materials of the above type, a liquid carrier such as a fatty oil.

Oral delivery systems can also include sustained-release delivery systems that improve the amount of drugs absorbed from the stomach and small intestine (into the blood stream) over time course. A number of sustained-release systems are known in the art.

Various other materials may be present as coatings or to modify the physical form of the dosage unit. For instance, tablets can be coated with shellac, sugar, or both. A syrup can contain, in addition to active ingredient, sucrose as a sweetening agent, methyl and propylparabens as preservatives, a dye, and flavoring such as cherry or orange flavor.

The active agent(s) may also be administered in injectable dosages by solution or suspension of these materials in a physiologically acceptable diluent with a pharmaceutical adjuvant, carrier or excipient. Such adjuvants, carriers and/or excipients include, but are not limited to, sterile liquids, such as water and oils, with or without the addition of a surfactant and other pharmaceutically and physiologically acceptable components. Illustrative oils are those of petroleum, animal, vegetable, or synthetic origin, for example, peanut oil, soybean oil, or mineral oil. In general, water, saline, aqueous dextrose and related sugar solution, and glycols, such as propylene glycol or polyethylene glycol, are preferred liquid carriers, particularly for injectable solutions.

These active compounds may also be administered parenterally. Solutions or suspensions of these active compounds can be prepared in water suitably mixed with a surfactant such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

For use as aerosols, the compounds of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

Transdermal formulations include, without limitation, a transdermal delivery system, typically in the form of a patch that contains a depot of the active drug(s) in a pharmaceutically acceptable transdermal carrier, or simply a solution phase carrier that is deposited onto the skin, where it is absorbed. A number of transdermal delivery systems are known in the art, such as U.S. Pat. No. 6,149,935 to Chiang et al., PCT Application Publ. No. WO2006091297 to Mitragotri et al., EP Patent Application EP1674068 to Reed et al., PCT Application Publ. No. WO2006044206 to Kanios et al., PCT Application Publ. No. WO2006015299 to Santini et al., each of which is hereby incorporated by reference in its entirety.

Implantable formulations include, without limitation, polymeric matrices in which the drug(s) to be delivered are captured. Release of the drug(s) can be controlled via selection of materials and the amount of drug loaded into the vehicle. implantable drug delivery systems include, without limitation, microspheres, hydrogels, polymeric reservoirs, cholesterol matrices, polymeric systems and non-polymeric systems, etc. A number of suitable implantable delivery systems are known in the art, such as U.S. Pat. No. 6,464,687 to Ishikawa et al., U.S. Pat. No. 6,074,673 to Guillen, each of which is hereby incorporated by reference in its entirety.

Preferred dosages of the PDE1 inhibitor are between about 0.01 to about 2 mg/kg, preferably 0.05 to about 1 mg/kg, most preferably about 0.05 to about 0.5 mg/kg. For example, vinpocetine is commercially available in 10 mg doses. Dosages for β-blockers, ACE inhibitors, angiotensin II receptor antagonists, β-agonists, and NSAIDs are well known in the art. However, it is expected that the dosages of these other active agent(s) can, under certain circumstances, be reduced when co-administered with a therapeutically effective amount of the PDE1 inhibitor.

### EXAMPLES

The following examples are provided to illustrate embodiments of the present invention but they are by no means intended to limit its scope.

### Example 1

### Determination of PDE1 Isoform Expression in the Heart and Cardiomyocyte

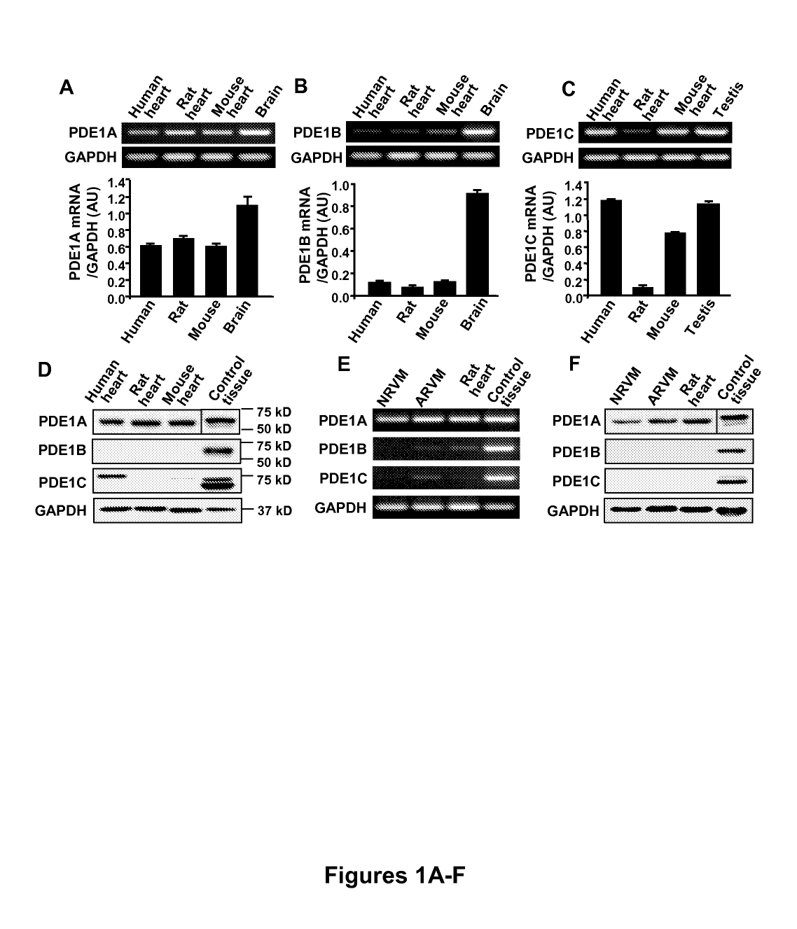

In human, rat, and mouse hearts, semi-quantitative RT-PCR analysis showed that PDE1A was detected at nearly equivalent levels in human, rat and mouse hearts, while PDE was primarily detected in human and mouse hearts, and PDE was weakly detected overall in the heart (FIGS. 1A-C). Western blotting analysis showed that PDE1A protein levels were comparable in hearts from different species whereas PDE1B was not detectable in the hearts, consistent with the mRNA expression (FIG. 1D). However, mouse heart elicited much lower PDE1C protein expression compared with human, inconsistent with the mRNA expression level (FIG. 1D). The low level of mouse heart PDE1C protein is unlikely a result of antibody insensitivity because the antibody strongly recognized mouse testis (FIG. 1D). In addition, PDE1A mRNA and protein in both NRVM and ARVM at a level comparable to that in adult rat heart (FIGS. 1E and F). In comparison, PDE1B and PDE1C expression levels were significantly lower in NRVM and ARVM. Together, these data indicate that both PDE1A and PDE1C isoforms are present in human hearts, while PDE1A expression is conserved in rodent hearts, particularly in rat cardiomyocytes.

### Example 2

### PDE1A Expression is Upregulated with Hypertrophic Stimulation In Vivo and in Isolated Cardiomyocytes In Vitro

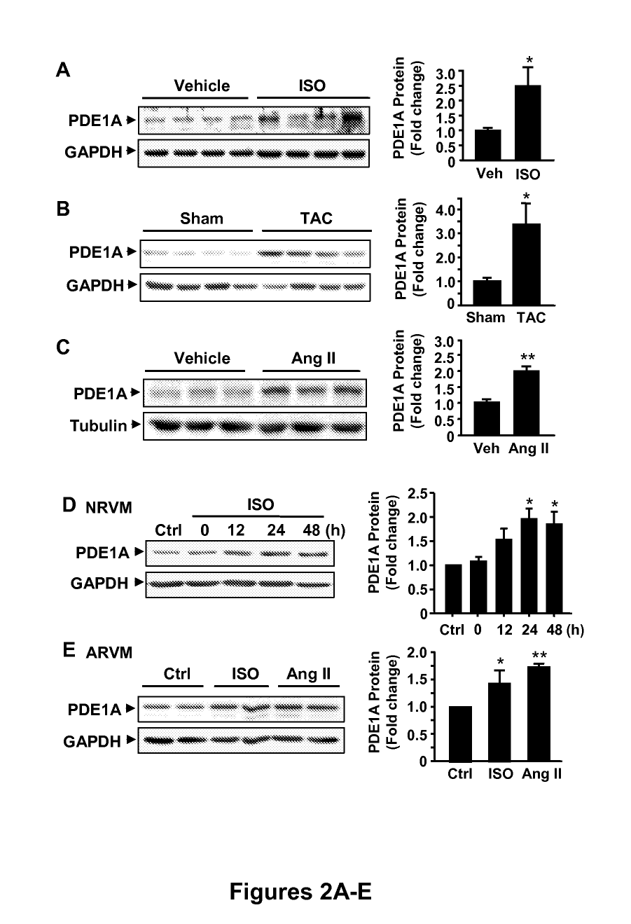

Western blotting analysis showed that PDE1A protein levels were significantly up-regulated in animal hypertrophied hearts, including mouse hearts with chronic isoproterenol (ISO) infusion (30 mg/kg/d for 7 days) (FIG. 2A); mouse hypertrophied hearts induced by chronic pressure overloaded via transverse aortic constriction (TAC) for 4 weeks (FIG. 2B); or rat hearts with chronic Ang II infusion (0.7 mg/kg/d for 7 days) via osmotic mini pump (FIG. 2C). These models are well-established rodent models of cardiac hypertrophy. In isolated NRVM, ISO treatment increased PDE1A protein levels relative (FIG. 2D). Similarly, ISO or Ang II treatment of ARVM resulted in an increase in PDE1A protein levels (FIG. 2E). Together, these data indicate that PDE1A expression can be upregulated in cardiomyocytes via hypertrophic stimuli both in vivo and in vitro. Western blots (left side panels) are quantified by densitometry (right side diagrams). Values were normalized to the control (Veh or Sham) that was arbitrarily set to 1.0. Data represent the mean of at least four samples (mean±SD.). GAPDH or tublin was used as equal loading control.

### Example 3

### Effects of PDE1 Inhibition On Cardiomyocyte Hypertrophic Growth

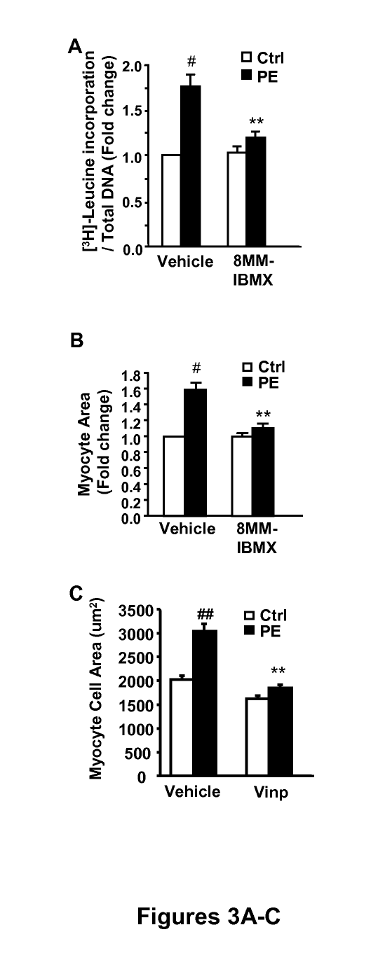

PDE1 inhibitor, 8-MM-IBMX (8-methoxymethyl-isobutylmethylxanthine) used at 20 μmol/L (the dose selectively inhibiting PDE1), significantly attenuated the PE-induced rat neonatal cardiomyocytes hypertrophy assessed by protein synthesis with ³H-leucine incorporation (FIG. 3A) or by myocyte surface area (FIG. 3B). Vinpocetine (20 μM), known as PDE1 inhibitor, also significantly reduced PE-induced myocyte hypertrophy measured by myocyte surface area (FIG. 3C). Rat neonatal cardiomyocytes were cultured in serum-free medium for 24 hours. Cells were pretreated with 20 μM 8-MM-IBMX or vehicle DMSO, followed by without (control, ctrl) or with PE treatment for 48 hours. Pulse chase of [³H]-leucine labeling was performed for the last 6 hours. Cells were lysed and ³H-leucine incorporation in cell lysates were then measured by scintillation counter. The values of ³H-leucine were normalized to DNA contents. Data were normalized to control (IC86340 at zero, without PE) that was arbitrarily set to 1.0. Data are means of triplicates (mean±SD). Similar results were obtained from at least three independent experiments. **p<0.01 vs. control (vehicle, without PE). #p<0.05 vs. with PE alone.

Effects of PDE1 inhibitor 8-MM-IBMX on PE-stimulated cell hypertrophic growth were measured by cell surface area (FIG. 3B). Rat neonatal cardiomyocytes were treated with either 20 μM 8-MM-IBMX or vehicle, followed without (ctrl) or with PE stimulation. Cells were stained for α-actinin (a cardiomyocyte specific marker) to exclude the contamination of cardiac fibroblasts. At lease 100 α-actinin positive cells were analyzed. Cell surface area was measured by Image J program.

Effects of PDE1 inhibitor vinpocetine on PE-stimulated cell hypertrophic growth were measured by cell surface area (FIG. 3C). Rat neonatal cardiomyocytes were treated with either 20 μM Vinpocetine (vinp) or vehicle, followed without (ctrl) or with PE stimulation. Cell surface area was measured as above. Data were normalized to control (vehicle, without PE) that was arbitrarily set to 1.0. Data are means of at least 100 cells (mean±SD). Similar results were obtained from at least three independent experiments. **p<0.01 vs. control. ## p<0.01, #p<0.05 vs. PE alone.

### Example 4

### Effects of PDE1A-Downregulation on Cardiomyocyte Hypertrophic Growth

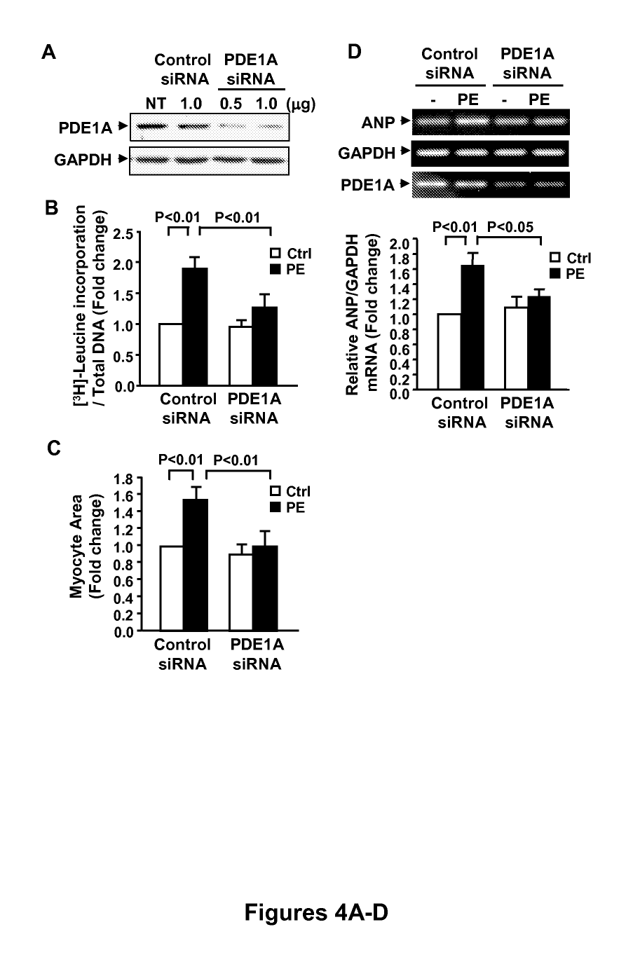

PDE1A protein levels were significantly reduced in rat neonatal cardiomyocytes transfected with PDE1A siRNA compared with control siRNA (FIG. 4A). Treatment with PDE1A siRNA significantly abrogated the PE-mediated increase in protein synthesis (FIG. 4B) and total myocyte surface area compared to control siRNA (FIG. 4C). Correspondingly, PDE1A siRNA also significantly attenuated PE-stimulated hypertrophic maker ANP expression (FIG. 4D). FIG. 4A illustrates a representative Western blot showing PDE1A protein expression in neonatal cardiomyocytes either not transfected (NT), or transfected with off-targeting control siRNA (1 μg) or rat PDE1A siRNA (0.5 or 1.0 μg) for 72 hours via electroporation. Similar results were observed in three independent experiments. Protein synthesis assessed by [³H]-leucine incorporation (normalized to the total DNA content) in NRVM transfected with 1 μg of control siRNA or PDE1A siRNA followed by PE (50 μmol/L) or vehicle (ctrl) stimulation for 48 hours (FIG. 4B). Data were normalized to the sample (with vehicle alone) that was arbitrarily set to 1.0. Values are mean±SD from six independent experiments (for siRNA) performed in triplicate. Total cell surface area of cardiomyocytes treated as mentioned above (FIG. 4C). The total cell surface area was averaged from 100 random alpha-actinin immuno-positive cells per condition. FIG. 4C illustrates representative RT-PCR results showing ANP and PDE1A mRNA expression in control or PDE1A siRNA treated myocytes with PE stimulation. Data were quantified by densitometry in a linear range and normalized to GAPDH mRNA levels. Values are mean±SD of three independent experiments.

### Example 5

### Role of Vinpocetine in Cardiomyocyte Hypertrophy Cardiac Hypertrophy In Vivo

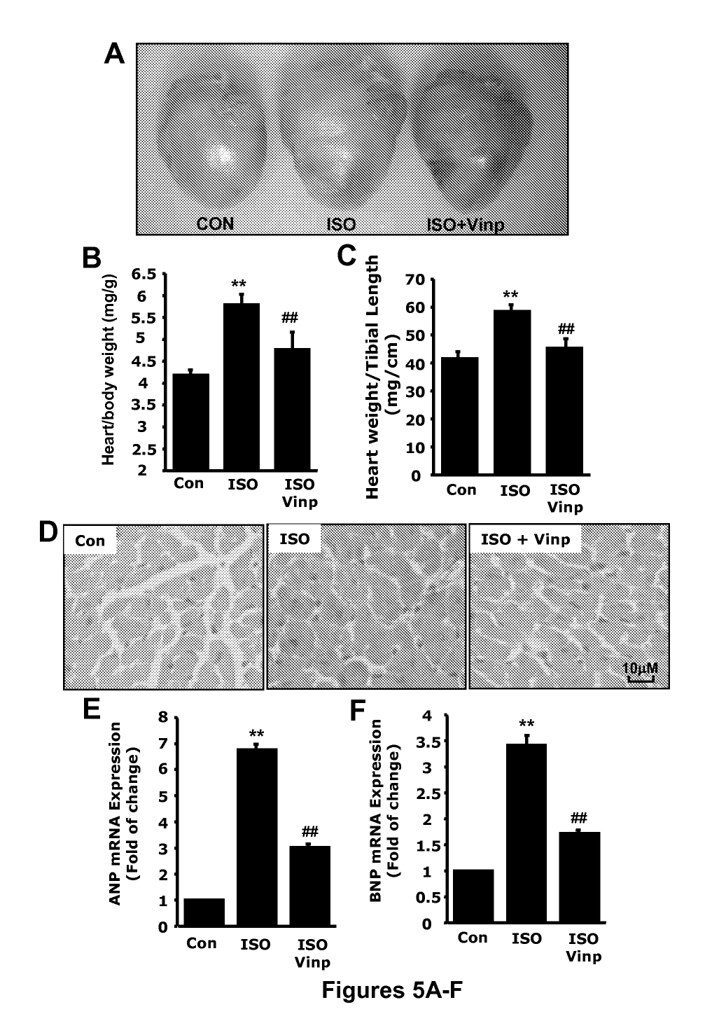

Since Vinpocetine has been widely used in many countries for preventative treatment of cerebrovascular disorder and cognitive impairment (Bonoczk et al., “Role of Sodium Channel Inhibition in Neuroprotection: Effect of Vinpocetine,” Brain Res Bull. 53:245-54 (2000), which is hereby incorporated by reference in its entirety), and it has been shown to be a safe for long-term use. PDE1 is a well known biological target for Vinpocetine. In vitro, Vinpocetine significantly blocked PE-induced cardiomyocyte hypertrophic growth, similar to other PDE1 inhibitors (FIG. 3). Based on these reasons, the effects of Vinpocetine were tested on cardiomyocyte hypertrophy in vivo. Excitingly, it was discovered that daily administration of Vinpocetine (i.p. 10 mg/kg/d) also significantly reduced mouse cardiac hypertrophy induced by chronic ISO infusion with osmotic pumps (30 mg/kg/d for 7 days) measured by gross heart morphology (FIG. 5A), heart weight/body weight (HW/BW) ratio (FIG. 5B), and heart weight/tibia length (HW/TL) ratio (FIG. 5C). To confirm the effect of Vinpocetine on ISO-induced cardiac hypertrophy, cross-section areas of cardiomyocytes in left ventricles were evaluated by hematoxylin and eosin staining Consistent with the increase in heart weight/body weight ratio, ISO infusion caused an increase in cross-section areas of cardiomyocytes in left ventricles compared with control (FIG. 5D, middle panel vs. left panel) and this effect of ISO infusion was significantly attenuated by the treatment of Vinpocetine (FIG. 5D, right panel vs. middle panel). Moreover, it was found that the mRNA levels of two hypertrophic makers, ANP (FIG. 5E) and BNP (FIG. 5F), were also significantly decreased in Vinpocetine treated heart samples. These results indicate that Vinpocetine may be an ideal and safe therapeutic agent for prevention of pathological cardiac remodeling and progression of heart failure.

### Discussion of Examples 1-5:

Vinpocetine, a derivative of alkaloid vincamine, has long been used in the clinic for the treatment of cerebrovascular disorder and cognitive impairment. Vinpocetine is well known to enhance cerebral circulation and cognitive function and is currently used as a dietary supplement in many countries for preventative treatment of cerebrovascular disorder and related symptoms associated with aging. Large clinical trials with vinpocetine indicate that vinpocetine dilates blood vessels, enhances circulation in the brain, enhances oxygen utilization and glucose uptake from blood and thus activates cerebral metabolism and neuronal ATP bio-energy production. In addition, Vinpocetine also elicits neuronal protection effects which increase resistance of the brain to hypoxia and ischemic injury. Vinpocetine was shown to easily cross the blood-brain barrier, which makes Vinpocetine one of the rather few drugs that exert a potent, favorable effect on the cerebral circulation.

The first molecular target identified for vinpocetine was Ca²⁺/calmodulin-stimulated phosphodiesterases (PDEs) (Bonoczk et al., “Role of Sodium Channel Inhibition in Neuroprotection: Effect of Vinpocetine,” Brain Res Bull. 53:245-54 (2000), which is hereby incorporated by reference in its entirety). PDEs, by catalyzing the hydrolysis of cAMP and cGMP, play critical roles in controlling intracellular cyclic nucleotide levels and compartmentation. PDEs constitute a large superfamily of enzymes grouped into 11 broad families based on their distinct kinetic properties, regulatory mechanisms, and sensitivity to selective inhibitors (Yan et al., “Functional Interplay Between Angiotensin II and Nitric Oxide: Cyclic GMP as a Key Mediator,” Arteriosclr Thromb Vasc Biol 23:26-36 (2003), which is hereby incorporated by reference in its entirety). Four major families of PDEs have been identified in VSMCs, including Ca²⁺/calmodulin-stimulated PDE1, cGMP-inhibited PDE3, cAMP-specific PDE4, and cGMP-specific PDES. The positive vascular effect in cerebral vasodilation of Vinpocetine is at least partially due to its effect on PDE1 inhibition.

Vinpocetine can be used for treatment or preventing pathological cardiac remodeling resulted from a variety of human diseases such as hypertension, myocardial infarction, diabetes, renal disease, and viral myocarditis. It can be used either alone or in conjunction with other drugs, such as β-blocker or Ang II receptor antagonists or ACE inhibitors, or even β-agonists. In the case of β-blocker, it may significantly reduce the dosage of β-blocker so that negative inotropic effect of using β-blocker can be minimized.

The present invention shows that PDE1, particular PDE1A, a molecular target existing in the cardiomyocyte, regulates cardiomyocyte hypertrophic growth. Vinpocetine, a clinically proven safe drug, showed potent anti-hypertrophic effect. The present invention demonstrates that PDE1, such as PDE1A, is a target for cardiac hypertrophy, and that Vinpocetine acts as a novel and potent anti-hypertrophic agent in vitro and in vivo. Vinpocetine has long been used for treatment of the cerebrovascular disorder and cognitive impairment. Vinpocetine has already been clinically approved to be safe and no significant side effects have been reported after long-term use. Therefore, vinpocetine should be an ideal therapeutic agent for treating the chronic disease, cardiac hypertrophy and heart failure.

Moreover, given the positive results achieved with Vinpocetine, it is believed that other PDE1 inhibitors, particularly PDE1A inhibitors, can also be utilized in the treatment or prevention of pathological cardiac remodeling and progression of heart failure.

### Example 6

### Combination Therapy for Treatment of Heart Failure

Patients diagnosed with heart failure will be administered daily dosage of the PDE1 inhibitor Vinpocetine (10 mg orally, three times daily) alone or in combination with the β-agonist terbutaline (5 mg, three times daily) or the PDE3 inhibitor Milrinone (10 mg, four times daily). The efficacy of the combination therapies will be compared to patients receiving Vinpocetine alone and placebo. Weekly assessment of efficacy will be made by measurement of the Oxygen Uptake Efficiency Slope during submaximal exercise (Hollenberg et al., “Oxygen Uptake Efficiency Slope: An Index of Exercise Performance and Cardiopulmonary Reserve Requiring only Submaximal Exercise,” J Am Coll Cardiol 36:194-201 (2000), which is hereby incorporated by reference in its entirety) and echocardiogram.

### Example 7

### Combination Therapy to Prevent Onset of Cardiac Remodeling

Patients diagnosed with heart failure will be administered daily dosage of the PDE1 inhibitor Vinpocetine (10 mg orally, three times daily) alone or in combination with the β-AR antagonist metoprolol (50 mg orally, three times daily). The efficacy of the combination therapies will be compared to patients receiving Vinpocetine alone and placebo. Weekly assessment of efficacy will be made by echocardiogram.

All of the features described herein (including any accompanying claims, abstract and drawings), and/or all of the steps of any method or process so disclosed, may be combined with any of the above aspects in any combination, except combinations where at least some of such features and/or steps are mutually exclusive. Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of treating or preventing pathological cardiac remodeling and/or heart failure comprising:
providing an inhibitor of phosphodiesterase 1 activity (“PDE1 inhibitor”); and
administering the PDE1 inhibitor to a patient under conditions effective to treat or prevent pathological cardiac remodeling and/or heart failure.

2. The method according to claim 1 wherein the PDE1 inhibitor is selected from the group consisting of a vincamine derivative, bepridil, flunarizine, amiodarone, 8-MM-IBMX, KS-505a, K-295-2, KS-619-1, IC86340, IC295, SCH51866, SCH45752, Schering Compound 30, Schering Compound 31, a ginsenoside, and anti-PDE1 RNAi.

3. The method according to claim 2 wherein the RNAi comprises siRNA, shRNA, or anti-sense PDE1 oligonucleotides.

4. The method according to claim 2 wherein the vincamine derivative is selected from the group consisting of 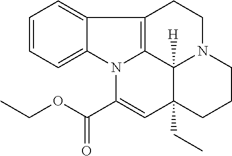
(+)-vinpocetine or salts thereof; 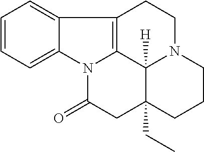
(−)-eburnamonine (also known as viburnine) or salts thereof; 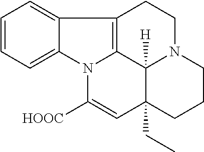
apovincaminic acid or salts thereof; 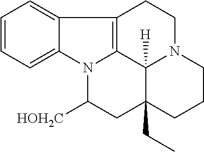
(3S,16R)-didydro-eburnamenine-4-methanol (also known as RGH-0537) or salts thereof; 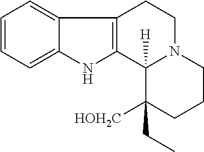
(1S,12S)-indoloquinolizinyl-1-methanol (also known as RGH-2981 or vintoperol) or salts thereof; 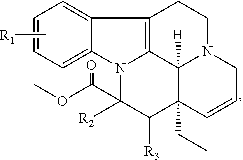
where R₁ is a halogen, R₂ can be a hydroxy group whereas R₃ can be hydrogen, or R₂ and R₃ together form an additional bond between the carbon atoms which carry them, or salts thereof; 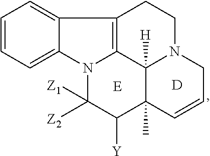
where the compound is formed by a cis-fusion of the D/E rings, and either (i) Y is hydrogen, in which case Z₁ and Z₂ together represent simultaneously an oxygen atom or Z₁ is a methoxycarbonyl radical and Z₂ is a hydroxy radical, or (ii) where Y and Z₂ together form a carbon-carbon bond and Z₁ is a methoxycarbonyl radical, or salts thereof; 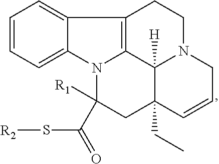
where R₁ is hydrogen or a hydroxyl group, and R₂ is an alkyl group, or salts thereof; 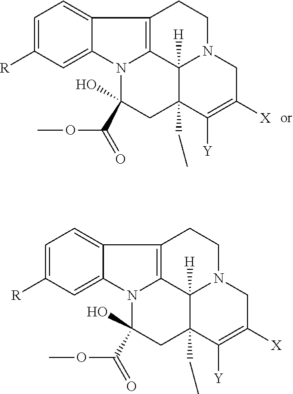
where R is hydrogen or methoxy, X and Y are hydrogen or are together are a double bond between the ring carbon atoms to which they are bonded, or salts thereof and
combinations of any two or more of the above compounds or salts thereof.

5. (canceled)

6. The method according to claim 1 wherein the administering is effective to treat symptoms of a pre-existing pathological cardiac remodeling.

7-8. (canceled)

9. The method according to claim 6 further comprising co-administering the PDE1 inhibitor with a β-agonist or an inhibitor of phosphodiesterase 3 activity (“PDE3 inhibitor”).

10. The method according to claim 1 wherein the administering is carried out prior to onset of pathological cardiac remodeling.

11-12. (canceled)

13. The method according to claim 10 further comprising co-administering the PDE1 inhibitor with a β-blocker.

14. (canceled)

15. The method according to claim 1 further comprising co-administering a therapeutically effective amount of an additional therapeutic agent to the patient, wherein the additional therapeutic agent is selected from the group of β-blockers, β-agonists, a PDE3 inhibitor, an angiotensin II receptor (type 1) antagonist, an angiotensin-converting enzyme (ACE) inhibitor, and a metabolism-boosting agent.

16-21. (canceled)

22. The method according to claim 1 wherein the patient is a mammal.

23. (canceled)

24. The method according to claim 1 wherein the administering is carried out orally, by inhalation, by airway instillation, optically, intranasally, topically, transdermally, parenterally, subcutaneously, intravenous injection, intra-arterial injection, intradermal injection, intramuscular injection, intrapleural instillation, intraperitoneal injection, intraventricularly, intralesionally, by application to mucous membranes, or implantation of a sustained release vehicle.

25. The method according to claim 1, wherein the PDE1 inhibitor is present in a pharmaceutical composition further comprising a pharmaceutically acceptable carrier.

26. The method according to claim 1 wherein the PDE1 inhibitor is administered in an amount of about 0.01 to about 2 mg/kg.

27. A pharmaceutical composition comprising a PDE1 inhibitor and either a β-blocker, a β-agonist, a PDE3 inhibitor, a metabolism-boosting agent, or a combination thereof.

28. The pharmaceutical composition according to claim 27, wherein the β-blocker is selected from the group consisting of acebutolol, atenolol, betaxolol, bisoprolol or bisoprolol fumarate, carvedilol, carteolol, celeprolol, esmolol or esmolol hydrochloride, labetalol, metoprolol or metoprolol succinate or metoprolol tartrate, nadolol, nebivolol, oxprenolol, penbutolol, pindolol, propranolol or propranolol hydrochloride, sotalol, esmolol, carvedilol, timolol, bopindolol, medroxalol, bucindolol, levobunolol, metipranolol, celiprolol, and propafenone.

29. The pharmaceutical composition according to claim 27, wherein the β-agonist is selected from the group consisting of dobutamine, formoterol or formoterol fumarate, fenoterol, ritodrin, salbutinol, terbutaline, isoproterenol, and clenbuterol.

30. The pharmaceutical composition according to claim 27, wherein the PDE3 inhibitor is selected from the group consisting of milrinone, aminone, enoximone, and combinations thereof.

31. The pharmaceutical composition according to claim 27 further comprising an angiotensin II receptor (type 1) antagonist and/or an angiotensin-converting enzyme (ACE) inhibitor.

32. The pharmaceutical composition according to claim 31 wherein the angiotensin II receptor (type 1) antagonist is selected from the group consisting of saralasin acetate, candesartan cilexetil, CGP-63170, EMD-66397, KT3-671, LR-B/081, valsartan, A-81282, BIBR-363, BIBS-222, BMS-184698, candesartan, CV-11194, EXP-3174, KW-3433, L-161177, L-162154, LR-B/057, LY-235656, PD-150304, U-96849, U-97018, UP-275-22, WAY-126227, WK-1492.2K, YM-31472, losartan potassium, E-4177, EMD-73495, eprosartan, HN-65021, irbesartan, L-159282, ME-3221, SL-91.0102, tasosartan, telmisartan, UP-269-6, YM-358, CGP-49870, GA-0056, L-159689, L-162234, L-162441, L-163007, PD-123177, A-81988, BMS-180560, CGP-38560A, CGP48369, DA-2079, DE-3489, DuP-167, EXP-063, EXP-6155, EXP-6803, EXP-7711, EXP-9270, FK-739, HR-720, ICI-D6888, ICI-D7155, ICI-D8731, isoteoline, KR1-1177, L-158809, L-158978, L-159874, LR B087, LY-285434, LY-302289, LY-315995, RG-13647, RWJ-38970, RWJ-46458, S-8307, S-8308, saprisartan, saralasin, Sarmesin, WK-1360, X-6803, ZD-6888, ZD-7155, ZD-8731, BIBS39, C1-996, DMP-811, DuP-532, EXP-929, L-163017, LY-301875, XH-148, XR-510, zolasartan, PD-123319, and combinations thereof.

33. The pharmaceutical composition according to claim 31 wherein the ACE inhibitor is selected from the group consisting of AB-103, ancovenin, benazeprilat, BRL-36378, BW-A575C, CGS-13928C, CL242817, CV-5975, Equaten, EU4865, EU-4867, EU-5476, foroxymithine, FPL 66564, FR-900456, Hoe-065, 15B2, indolapril, ketomethylureas, KR1-1177, KR1-1230, L681176, libenzapril, MCD, MDL-27088, MDL-27467A, moveltipril, MS41, nicotianamine, pentopril, phenacein, pivopril, rentiapril, RG-5975, RG-6134, RG-6207, RGH0399, ROO-911, RS-10085-197, RS-2039, RS 5139, RS 86127, RU-44403, S-8308, SA-291, spiraprilat, SQ26900, SQ-28084, SQ-28370, SQ-28940, SQ-31440, Synecor, utibapril, WF-10129, Wy-44221, Wy-44655, Y-23785, Yissum, P-0154, zabicipril, Asahi Brewery AB-47, alatriopril, BMS 182657, Asahi Chemical C-111, Asahi Chemical C-112, Dainippon DU-1777, mixanpril, Prentyl, zofenoprilat, I (-(1-carboxy-6-(4-piperidinyl)hexyl)amino)-1-oxo-propyl octahydro-1H-indole-2-carboxylic acid, Bioproject BP1.137, Chiesi CHF 1514, Fisons FPL-66564, idrapril, perindoprilat and Servier S-5590, alacepril, benazepril, captopril, cilazapril, delapril, enalapril, enalaprilat, fosinopril, fosinoprilat, imidapril, lisinopril, perindopril, quinapril, ramipril, ramiprilat, saralasin acetate, temocapril, tranolapril, trandolaprilat, ceranapril, moexipril, quinaprilat, spirapril, and combinations thereof.

34. The pharmaceutical composition according to claim 27 wherein the metabolism-boosting agent is selected from the group of coenzyme A, ATP, coenzyme Q10 (CQ10), NAD(P)H, and insulin-like growth factor-1 (IGF-1).

35. The pharmaceutical composition according to claim 27 wherein the PDE1 inhibitor is selected from the group consisting of a vincamine derivative, bepridil, flunarizine, amiodarone, 8-MM-IBMX, KS-505a, K-295-2, KS-619-1, IC86340, IC295, SCH51866, SCH45752, Schering Compound 30, Schering Compound 31, a ginsenoside, and anti-PDE1 RNAi.

36. The pharmaceutical composition according to claim 27 further comprising a pharmaceutically acceptable carrier.

37-39. (canceled)

40. A delivery vehicle comprising the pharmaceutical composition according to claim 27, wherein the delivery vehicle is in the form of a transdermal patch, a syringe, or a biocompatible polymeric matrix

41. A method of preventing heart failure comprising:
providing an inhibitor of phosphodiesterase 1 activity (PDE1 inhibitor); and
administering the PDE1 inhibitor to a patient susceptible to pathological cardiac remodeling under conditions effective to prevent heart failure caused by pathological cardiac remodeling.

42. The method according to claim 41 wherein the PDE1 inhibitor is selected from the group consisting of a vincamine derivative, bepridil, flunarizine, amiodarone, 8-MM-IBMX, KS-505a, K-295-2, KS-619-1, IC86340, IC295, SCH51866, SCH45752, Schering Compound 30, Schering Compound 31, a ginsenoside, and anti-PDE1 RNAi.

43. The method according to claim 41 further comprising co-administering a therapeutically effective amount of an additional therapeutic agent to the patient, wherein the additional therapeutic agent is selected from the group of β-blockers, β-agonists, a PDE3 inhibitor, an angiotensin II receptor (type 1) antagonist, an angiotensin-converting enzyme (ACE) inhibitor, and a metabolism-boosting agent.

44. The method according to claim 41 wherein the patient is a mammal.

45. The method according to claim 41, wherein the administering is carried out orally, by inhalation, by airway instillation, optically, intranasally, topically, transdermally, parenterally, subcutaneously, intravenous injection, intra-arterial injection, intradermal injection, intramuscular injection, intrapleural instillation, intraperitoneal injection, intraventricularly, intralesionally, by application to mucous membranes, or implantation of a sustained release vehicle.

46. The method according to claim 41, wherein the PDE1 inhibitor is present in a pharmaceutical composition further comprising a pharmaceutically acceptable carrier.

47. The method according to claim 41, wherein the PDE1 inhibitor is administered in an amount of about 0.01 to about 2 mg/kg.
